(19) United States
(12) Patent Application Publication
(10) Pub. No.: US 2023/0323264 A1
(43) Pub. Date: Oct. 12, 2023

(54) Artificial lymph node bioreactor

(71) Applicant: Mirror Biologics, Inc., Lutz, FL (US)

(72) Inventor: Michael Har-Noy, Jerusalem (IL)

(21) Appl. No.: 18/131,112

(22) Filed: Apr. 5, 2023

Related U.S. Application Data
(60) Provisional application No. 63/328,382, filed on Apr. 7, 2022.

(51) Int. Cl. C12M 3/00; C12M 1/00

(52) U.S. Cl. CPC C12M 23/48; C12M 29/10; C12M 23/02

(57) ABSTRACT

A system and method for high density cell culture support utilizing two circulation circuits: a cell culture loop and a media conditioning loop.

### CROSS-REFERENCE TO RELATED APPLICATION

The present application claims priority to U.S. patent application Ser. No. 63/328,382, filed Apr. 7, 2022, the contents of which is hereby incorporated by reference in its entirety.

### BACKGROUND

The present invention relates to an artificial lymph node bioreactor system. More specifically the system is designed for expansion and maintenance of high-density mammalian cell culture, in particular primary immune cells, which utilizes two circulation circuits: a cell culture loop and a media conditioning loop integrated with a cell separation device.

### SUMMARY

This disclosure describes a system for high density cell culture support comprising a first circulation loop comprising a cell containing loop and a first holding container and one or more pumps for continuous circulation of cell containing media and a second circulation loop that is a cell-free media conditioning loop and a second holding container and one or more pumps for circulating media; and a controller for directing the circulation of a first quantity of media through the first circulation loop and a second quantity of media through the second circulation loop.

In another embodiment the system comprises a cell separation device between the first circulation loop and the second circulation loop.

In another embodiment the system the cell separation device is configured to use centrifugal force to continuously separate cells from media.

In another embodiment the system the second holding container is operably connected to the cell separation device and wherein the cell separation device is configured to return concentration cells to the first holding container for completing the first circulation loop.

In another embodiment the system the cell separation device is configured to deliver separated cell-free media to the second holding device for reconditioning the media, and wherein the reconditioned media is then returned to the first holding device for completing the second circulation loop.

In another embodiment wherein the second holding container comprises a circulation loop configured to delivery cell-free media through a lumen side of a hollow fiber artificial lung and wherein the artificial lung loop is configured for oxygenating the cell-free media in the second holding container.

In another embodiment the system the second holding container comprises one or more sensors for continuous measurement of at least one of pH, oxygen, glucose lactate, ammonia and combinations thereof.

In another embodiment wherein the first circulation loop comprises one or more probes for measuring pH of media, dissolved oxygen, or a combination thereof of media inside the first holding container and/or wherein the first holding container is configured to continuously monitor a weight of the container which is correlated to a volume of media within the first holding container.

This disclosure also describes a method for high density cell culture support comprising: concentrating a mass of mammalian cells in a first holding container and separating the concentrated mass by removing cell-free media from the mass of concentrated mammalian cells in a first circulation loop; delivering a mass of the cell free media to a second circulation loop; conditioning the mass of cell free media in a second holding container in the second loop; removing waste products from the mass of cell free media in the second loop; and returning the conditioned mass of cell free media to the first circulation loop.

In another embodiment of this method the conditioning in the second holding container comprises reoxygenating the cell free media in a high-speed recirculation loop through an artificial lung hollow fiber cartridge device.

In another embodiment of this method the conditioning further comprises adjusting one or more metabolic parameters comprising pH, glucose, lactate, glutamine, ammonia or combinations thereof.

In another embodiment of this method the cells are separated in the first container from the spend media is a continuous process and further comprises using centrifugal force to remove fractions of cell free media from the cells in one of a continuous or semi-batch process.

In another embodiment of this method the conditioned media is returned from the second holding container to the first holding container replaces the volume of waste media removed from the cells in the centrifugation device.

In another embodiment of this method the cells are separated in a centrifuge device between the first circulation loop and the second circulation loop by adjusting one or more of the retention time of the cells in the centrifuge, the speed (rpm) of the centrifuge, the rate of a counter elutriating pump, and adjusting a recirculation rate of the cells to maintain a predetermined level of media in the first holding container.

In another embodiment of this method the method comprises extracting a sample of cell free media from the second holding container; analyzing one or more of glucose, glutamine, ammonia, lactic acid or osmolarity; and adjusting one or more of glucose, glutamine, ammonia, lactic acid or osmolarity in response to analysis by adjustment of one or more parameters of a media, glucose and glutamine delivery pumps or a waste removal pump.

In yet another method, this disclosure includes a combined batch-feed and perfusion culture method comprising carrying out a first batch-feed process for seeding cells at a first concentration, allowing the cells to grow to a first volume, and adjusting the cell concentration back to the first concentration by adding conditioned media, and repeating the batch feed process in an integrating vessel until a first selected volume is reached; engaging a first and second circulation loop to perfuse cells in the integrating vessel and increasing cell density within the first selected volume; monitoring cell number and cell viability through samples taken from the circulation loop through a sampling port in the integrating vessel; and adding conditioned media from the second circulation loop for maintaining a constant volume.

In another embodiment of the method of a combined batch-feed and perfusion culture method wherein perfusion comprises: initiating a perfusion cycle including moving cells from the integrating vessel to a spinning container creating centrifugal force and separating cell-free media from the cells; returning heavier cells to the integrating vessel; and delivering cell free media to a second holding container in a second circulation loop for conditioning of the cell-free media.

In another embodiment of the method of the combined batch-feed and perfusion culture method engaging a dialysis loop as a part of the second holding container using a hollow fiber cartridge to remove lactic acid and other metabolic wastes from the cell free media.

In another embodiment of the method of the combined batch-feed and perfusion culture method and further including continuously monitoring an oxygen level of the cell-free media and if the oxygen level falls below a pre-selected set-point, increasing the rate of delivery of conditioned media concurrently with proportionally increasing a rate of cell removal and waste removal from the first circulation loop.

In another embodiment of the method of the combined batch-feed and perfusion culture method and further including adjusting pH in the second vessel using a proportional-integral-derivative controller (PID) control loop for lowering concentration of CO₂ in the artificial lung and replacing the CO₂ with air or N₂ gas; and when a CO₂ lever reaches zero, adding fresh cell free media or buffer to the second holding container to raise the pH of the cell free media.

### DRAWINGS

FIG. 1 is a schematic view of an embodiment of a system according to one or more embodiments described herein.

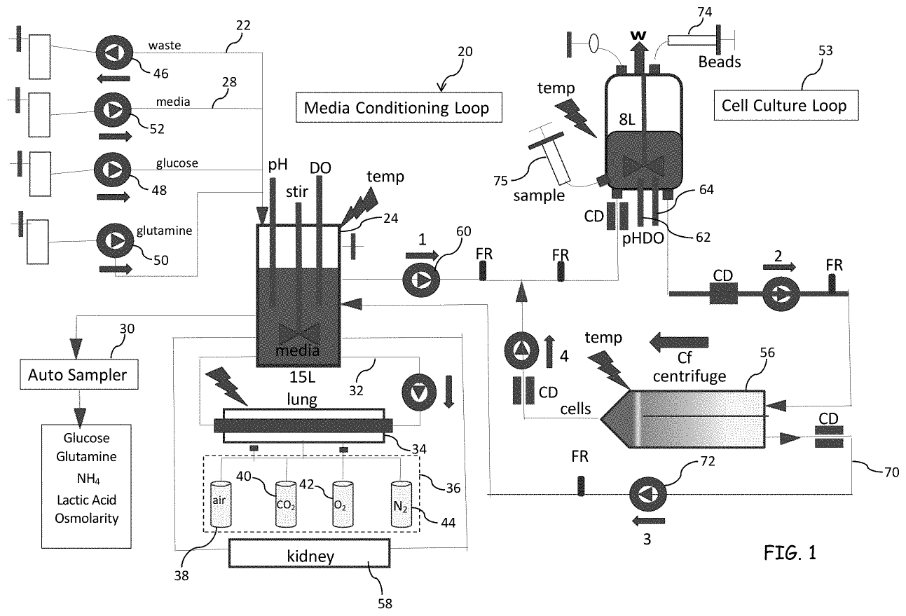

In FIG. 1, terms are defined as follows: FR=flow rate meter, CD=infrared cell density sensor, W=weight, and DO=dissolved oxygen. In the embodiment of FIG. 1, a first pump, Pump 1, is a perfusion pump. The pump rate will increase to maintain DO at a set point in the cell culture loop 53 (pump 1+4=pump 2). The rate for Pump 2=rate pump 3+rate pump 4.

### DETAILED DESCRIPTION

The system may be a bioreactor with major components that include two holding containers and the two separate circulation loops. The two holding containers comprise a cell-free holding container for conditioning of culture media to adjust metabolic parameters (e.g., pH, glucose, lactate, glutamine and ammonia) and for oxygen saturation using a high-speed hollow fiber oxygenator; and a bioreactor container for culturing of mammalian cells to high density (i.e., >10⁷ cells/ml). The cell-free container has a circulating loop for continuous delivery of oxygen saturated and metabolically adjusted media to the bioreactor container. The bioreactor container has a continuous cell recirculation loop that separates cells and waste media using centrifugal force, returning cells to the bioreactor, and the waste media from the bioreactor loop is returned to the cell-free container for re-conditioning. Re-conditioned media from the cell-free loop is returned to the bioreactor container to replace the waste media that was removed from the cell recirculation loop.

The bioreactor described herein is superior to other bioreactors as it solves the problem of insufficient dissolved oxygen transfer rate to mammalian cells in culture, a limitation which prevents the ability to support high density cell cultures.

In some embodiments, the bioreactor may support cell densities over about 10 million cells per ml. In one embodiment, the bioreactor may support cell densities of about 100 million per ml. These cell densities can be supported at volumes of up to 8 liters in some embodiments and 10 liters or more in one embodiment.

An aspect of the present disclosure relates to a system for high density cell culture support of cells >1×10⁷ ml. The system has a first circulation loop that is a cell containing loop and comprising a first holding container and one or more pumps for continuous circulation of cell containing media and a second circulation loop that is a cell-free media conditioning loop and comprising a second holding container and one or more pumps for circulating media. A controller is provided for directing the circulation of a first quantity of media through the first circulation loop and a second quantity of media through the second circulation loop.

Between the first circulation loop and the second circulation loop is a cell separation device. The cell separation device preferably uses centrifugal force to continuously separate cells from media. Concentrated cells are returned to the first holding container while a counter elutriation force withdraws cell-free media and delivers to the second holding container.

The first holding container is operably connected to the cell separation device. The cell separation device returns concentrated cells to the first holding container completing the first circulation loop.

The cell separation device delivers the separated cell-free media to the second holding container where the media is re-conditioned. The re-conditioned media is then returned to the first holding device completing the second circulating loop.

The second holding container comprises a circulating loop that delivers cell-free media through the lumen side of a hollow-fiber artificial lung. Controlled mixtures of gases, including oxygen, air, carbon dioxide and nitrogen are delivered to the lumen side of the hollow-fiber artificial lung by circulating the media from the second vessel at high speed through the hollow fiber artificial lung, the dissolved oxygen concentration can reach saturation in a short period of time. In this manner, the artificial lung loop provides for oxygenating the cell-free media in the second holding container and correction of the media pH. In addition, controlled rate pumps are used to deliver glucose and/or glutamine and fresh media to the second holding container while an additional pump is programmed to remove waste media from the second holding container in a manner that maintains a constant volume in the second container. An addition loop can be added to circulated the cell-free media from the second container to the lumen side of a dialysis hollow fiber device in order to remove lactate and ammonia waste products. The waste media is removed from the extra-capillary side of the dialysis device.

The second holding container contains sensors for continuous measurement of pH, oxygen, glucose, lactate and ammonia. Information from these sensors is used to program the pumps and gas flow connected to the second holding container to maintain programmed set points.

The first circulation loop comprises one or more probes for measuring pH of media, dissolved oxygen, or a combination thereof of media inside the first holding container. In addition, the first holding container is suspended on a system to constantly monitor the weight, which correlates with the volume of media within the container.

A temperature control system maintains the first holding container, first circulating loop, second holding container, cell separation device and second circulating loop at a controlled temperature set point, preferably 37° C.+/−3° C.

Another aspect of the present disclosure relates to a method for high density cell culture support. Cells normally reach densities of around 1×10⁶/ml in culture where they become oxygen limited to expand further. In addition, cells can only be maintained at densities of 1×10⁶/ml for a few days, as over time they consume all the available nutrients and produce toxic waste products. Oxygen concentration is due to the oxygen transfer rate of a gas into liquid, when the cell density reaches around 1×10⁶ cells/ml the oxygen uptake rate of the cells exceeds the oxygen transfer rate of oxygen into the liquid phase.

The oxygen uptake rate of an activated T-cells is ˜100 mmol/10¹⁰ cells/day or 0.1 mol/10¹⁰ cells/day. 10⁻⁵ mol/10⁶ cells/ml/day at 4×10⁻⁵ mol O₂/ml. Therefore, all O₂ is consumed in 4 days (assuming fully saturated media at time zero). If the cell density were increased to 1×10⁷ cells/ml then 10⁻⁴ mol/10⁷ cells/ml/day all O₂ consumed in 0.4 days (9.6 h). If the cell density were increased to 1×10⁸ cells/ml then 10⁻³ mol/10⁸ cells/ml/day all O₂ is consumed in 0.04 days (0.96 h). In order to supply sufficient oxygen to 4 L of cells at 1×10⁶/ml it would require a perfusion rate of 4 L/4 days=1 L/day or 42 ml/hr. At 10⁷/ml=4 L/0.4 day, 10 L/day, or 420 ml/hr. At 10⁸/ml=0.4 L/0.04 100 L/day or 4200 ml/hr or 70 ml/min.

Therefore, in a bioreactor there must be homogenous oxygen in the vessel. In order to supply oxygen at the rate it is consumed at a density of 10⁸/ml it requires the entire volume of the reactor to be exchanged at about 100× per hour.

To overcome this limitation, the second container with the high-speed artificial lung maintains a source of oxygen saturated media to deliver to the first container containing cells. The high speed necessary to rapidly saturate oxygen is not compatible with cells. Any cells in the high-speed circulation would be destroyed by shear. This the separation of the first container with cells and the second container without cells solves the problem by providing a ready source of oxygen saturated media to deliver to the first container with cells.

The method includes reconditioning the media in the second container with energy sources, such as glucose and glutamine and removing waste such as lactic acid and ammonia in order to solve the problem of depletion of nutrients and accumulation of waste products over time. This method decreases the total amount of media required to support cells in high density culture.

The cell separation device delivers cell containing media into a centrifugal force field designed to allow the high-density cells to continuously traffic through the first circulation loop without pelleting. A counter elutriation force, such as provided by a pump, in the opposite direction of the centrifugal force will first pull cell free media to the exit port. This pump will create a force slightly greater than the centrifugal force. A detector device, such as a photon light scatter detector at the exit port will detect any cells that traffic to the exit port. When cells are detected, the counter elutriation pump is slowed slightly allowing the cells to return to the first circulation loop. This process of speeding up and slowing down the elutriation pump will enable continuous recirculation of cells in the first circulation loop and removal of cell-free media for the second recirculation loop.

Conditioning in the second container includes reoxygenating the cell free media in a high-speed recirculation loop through an artificial lung hollow fiber cartridge device. Conditioning further includes adjusting one or more metabolic parameters selected from the group comprising pH, glucose, lactate, glutamine, ammonia and combinations thereof.

Separating the mammalian cells in the first container from spent media is a continuous process and the method includes using centrifugal force to remove fractions of cell free media from the mammalian cells in a continuous or semi-batch process.

Returning of the conditioned media from the second holding container to the first holding container replaces the volume of waste media removed from the cells in the centrifugation device.

The method also includes separating the mammalian cells in a centrifuge device between the first circulation loop and second circulation loop by adjusting one or more of the retention time of the cells in the centrifuge, the rpm of the centrifuge, the rate of the counter elutriating pump, and adjusting a recirculation rate of the cells to maintain a predetermined level of media in first holding container. The same amount of cell-free spent media removed from the separation device is returned as re-conditioned media to the first container.

In one or more embodiments, the method includes extracting a sample of cell free media from the second holding container; analyzing one or more of glucose, glutamine, ammonia, lactic acid and osmolarity; and adjusting one or more of glucose, glutamine, ammonia, lactic acid and osmolarity in response to analysis by adjustment of one or more parameters of a media, glucose and glutamine delivery pumps and a waste removal pump.

Yet another aspect of the present disclosure relates to a combined batch-feed and perfusion culture method. The method includes carrying out a first batch-feed process to seed cells at a concentration of 5×10⁵ cells/ml and allow to grow cells to around 1×10⁶ cells/ml in a first volume of for example 1 L. Subsequently adjust a cell concentration back to 5×10⁵ ml by adding an additional 1 L of conditioned media. This process of batch feed is continued until the desired volume is achieved. Once the desired volume is achieved in the first container, the first and second circulation loops are engaged to perfuse the cells in the first container and increase the cell density within the final volume achieved.

The cell concentration in the first vessel is monitored using in-line cell density detectors r and cell viability through samples taken from the circulation loop through a sampling port in the first vessel; adding conditioned media from the second circulation loop to balance the cell-free spent media removed in the secondary circulation loop in order to maintain a constant volume.

The perfusion steps include initiating a perfusion cycle including moving cells from the integrating vessel to a spinning container creating centrifugal force and separating cell free media from the cells; returning heavier cells to the first container; delivering the cell free media to a second holding container in a second circulation loop for conditioning of the cell free media.

The method can further include engaging a dialysis loop as part of the second vessel using a hollow fiber cartridge to remove lactic acid and other metabolic wastes from the cell free media.

Continuously monitoring a saturated oxygen level of the cell free media allows for adjustments, for example, if the oxygen level falls below a pre-determined set-point, increasing the rate of delivery of conditioned media concurrently with proportionally increasing a rate of cell removal and waste removal from the first circulation loop adjusts saturated oxygen levels.

pH in the second container can be adjusted to a set point using a proportional-integral-derivative controller (PID) control loop that lowers the concentration of CO₂ in the artificial lung loop and replacing with air or N₂ gas. When the CO₂ level reaches zero, adding fresh cell free media to the second holding container or buffer can raise the pH of the cell free media.

Media Conditioning Loop (MCL)

The media conditioning loop (MCL) 20 connects the waste return sub-loop 22 to the cell-free container 24 and the bioreactor container 26. The cell-free container 24 can include a 1-15 liters stirred bioreactor or flexible bag. The cell-free container 24 can be controlled to approximately 37° C. The MCL 20 also includes continuous monitoring of dissolved O₂ and pH and other probes that can monitor metabolic changes with sterile indwelling probes or external devices. The MCL 20 can include a sample port 30 for aseptic removal of media 28 for off-line measurement of glucose, lactate, NH₄ and osmolarity and other metabolic parameters. A sterile 0.2-0.45-micron air filter for pressure equalization can also be present. The MCL 20 may also include a continuous adjustable-rate high speed oxygenation loop 30 removing media from the cell-free container 24 and circulating media through the lumen of a hollow fiber oxygenator 34 and returning oxygenated media to the cell-free container 24. Gas is delivered through the extra capillary space of the oxygenator cartridge 34. The cartridge 34 is warmed to prevent condensation. Mass flow controllers (MFC) 36 control delivery of gases. In a preferred embodiment, there are 4 MFC with one each for air 38, CO₂ 40, O₂ 42 and N₂ 44.

For conditioning of the culture media in the cell-free container 24, controlled rate pumps can deliver nutrients and remove waste products. In a preferred embodiment, four controlled rate pumps are used for waste collection 46, glucose delivery 48, glutamine delivery 50 and media delivery 52. In one embodiment, an additional high-speed loop through a dialysis cartridge 58 can be used in order to retain cytokines, serum and growth factors while selectively removing metabolic waste, such as lactic acid and ammonia.

Cell Culture Loop (CCL)

The cell culture loop 53 incorporates a continuous centrifuge device 56 and connects the bioreactor container 26 thereto. The centrifuge device 56 concentrates cells and removes cell-free media. In one embodiment, the cell free media is delivered to the media conditioning loop 20 which re-oxygenates the media using an artificial lung 34 and removes waste with an artificial kidney dialysis device 58. External sampling and analysis of glucose, glutamine, ammonia, lactic acid and osmolarity are controlled through algorithms connected to media, glucose and glutamine delivery pumps and a waste removal pump. A 4 gas mass transfer system may be used to control pH and oxygen levels.

Media is delivered from the MCL 20 to the bioreactor 26 and the delivery may be controlled by an adjustable-rate pump 60. Sterile indwelling probes for pH 62 and dissolved oxygen (DO) 64 are inside the bioreactor 26. The contents are controlled at adjustable temperatures. The bioreactor 26 includes controlled valve ports for air pressure equalization or movement to the centrifuge 56, aseptic sampling ports 75 for offline measurements of cell count and viability, phenotype analysis, and a port 74 for aseptic adding of microbeads. The continuous centrifuge device 56 continuously separates cells from media, returning cells to the bioreactor 26 and waste media to the cell-free container. Media is delivered to the cell culture loop 53 to the bioreactor and returned to the media conditioning loop 20 from a port opposite the direction of the centrifugal force.

The bioreactor 26 can be a 1 liter or greater fermenter or gas permeable bag and in one embodiment the bioreactor may be a spherical 8 liter bioreactor. Cell containing media is removed from the bioreactor continuously and passed through a controlled centrifugal force field 56 which is designed to maintain continuous cell circulation out of the bioreactor 26 and back to the bioreactor 26. The centrifugal force field 56 is adjusted so as to separate cell-free media from the cell containing media. The cell-free media 70 is returned to the cell-free container 24 for reconditioning using a pump 72 that provides a force opposite the centrifugal force vector of the centrifuge 56

Discussion of Process Control loops

In one embodiment, a combined batch-feed and perfusion culture method is used. Approximately 50 million cells are transferred to the bioreactor 26 container using an aseptic injection port. Fresh oxygenated media from the integrating vessel or media conditioning loop (MCL) 20 the cell-free container 24 is transferred to the bioreactor 26 to adjust cell concentration to approximately 0.5×10⁶ cells/ml. The volume can be monitored by a digital weight sensor. In some embodiments, monoclonal antibody-coated microbeads or other growth factors are injected through an aseptic port 74 in the bioreactor.

Cell number and viability are monitored daily through samples taken from the aseptic sampling port 75. The cells are allowed to incubate undisturbed for 3 days or until the cell density reached 1×10⁶ cells/ml.

When the cell density reaches 1×10⁶ cells/ml, additional conditioned media from the cell-free container 24 is added by pump 60 to the bioreactor 26 to dilute the cell density to 0.5×10⁶ cells/ml. This fed-batch process is repeated each day until the total volume in the bioreactor reaches a pre-determined (selected) level. In a preferred embodiment, this level is 8 liters.

In another embodiment, CD3/CD28-coated microbeads are added through the aseptic port 74 every 3 days to maintain a 1:1 bead:cell ratio.

When volume reaches the pre-determined (selected) amount, the perfusion cycle is initiated. Cells are moved by pump from the bioreactor 26 to a spinning container creating centrifugal force. The heavier cells and beads will accumulate in the direction of the force vector and be returned to the bioreactor container. A pump that creates a force opposite of the centrifugal force vector will remove cell-free media and return the media to the cell-free container. The opposite vector force pump will oscillate so as to remove maximum media and release before any cells are removed. This may be accomplished by closing the vent valve and engaging the pump to transfer fresh conditioned media from the integrating vessel to the bag. Opening the valve between the bag and centrifuge will allow the cell/beads to flow into the centrifuge. The centrifugal force will retain the cells and beads and the media can be removed and returned to the integrating vessel for conditioning.

The retention time in the centrifuge and media removal rates can be adjusted by toggling the valves and adjusting the flow rate on the return pump. The rate of recirculation of the cells will increase to maintain the oxygen set point near 100% saturation in the bioreactor.

As the cell density in the bioreactor increases, lactic acid will accumulate driving the pH down.

To maintain pH at a set-point (generally between 6.8-7.4), the gas mixture delivered to the hollow-fiber oxygenator is adjusted with a PID control loop such that as the pH falls the percentage of CO₂ is lowered. CO₂ generally is set at 5% at the start of a process and is lowered as the pH falls.

When the pH controller calls for CO₂ levels less than 0%, the waste pump is engaged to remove media from the cell-free container and fresh media is pumped in to replace the media that was removed. In one embodiment, a dialysis loop is engaged using a 6000 dalton cut-off hollow fiber cartridge to remove lactic acid instead of removing and replacing complete media.

The rate of reconditioned media delivery to the bioreactor from the cell-free container will increase logarithmically as a function of the saturation oxygen level in the bioreactor. The saturated oxygen level is monitored continuously using a sterile probe in the bioreactor. As the level falls below a set-point (generally >95%) the rate of delivery of oxygenated media is increased. Simultaneously, the rate of cell removal and waste removal from the bioreactor to the centrifugal force device is increased proportionately.

The integrating chamber has a high-speed loop which pumps media through the lumen side of a hollow fiber oxygenator. The higher the speed the more oxygen can be dissolved per unit time. Since the media conditioning loop (MCL) is cell-free there is no shear issue that limits the pump speed. Increasing the stir rate can also increase the rate of oxygen transfer. In addition, the gas mixture can be adjusted to increase the percent O₂.

As the cells grow, the cells produce lactic acid which decreases the pH of the media. The initial gas mixture in the shell side of the hollow fiber oxygenator is 5% CO₂ in air. As the pH falls to a set point (e.g., 6.9), the percent CO₂ is lowered and replaced with nitrogen and oxygen mixture so the air flow rate and percent O₂ is constant. Eventually, the CO₂ level will reach 0. At zero CO₂, new media is added to dilute the lactate acid and bring the pH up. Glucose and glutamine are measured offline with samples from the aseptic port. Set points are established so glucose and glutamine are added, and waste removed via the controlled pumps to maintain set-point levels. If offline osmolarity reached a set point, the glucose and glutamine add pumps are over-ridden and fresh media is added.

Although the present disclosure has been described with reference to preferred embodiments, workers skilled in the art will recognize that changes may be made in form and detail without departing from the spirit and scope of the disclosure.

What is claimed:

1. A system for high density cell culture support comprising:
a first circulation loop comprising a cell containing loop and a first holding container and one or more pumps for continuous circulation of cell containing media;
a second circulation loop that is a cell-free media conditioning loop and a second holding container and one or more pumps for circulating media; and
a controller for directing the circulation of a first quantity of media through the first circulation loop and a second quantity of media through the second circulation loop.

2. The system of claim 1 and further comprising a cell separation device between the first circulation loop and the second circulation loop.

3. The system of claim 1 wherein the cell separation device is configured to use centrifugal force to continuously separate cells from media.

4. The system of claim 2 wherein the second holding container is operably connected to the cell separation device and wherein the cell separation device is configured to return concentration cells to the first holding container for completing the first circulation loop.

5. The system of claim 2 wherein the cell separation device is configured to deliver separated cell-free media to the second holding container for reconditioning the cell-free media, and wherein reconditioned media is returned to the first holding container for completing the second circulation loop.

6. The system of claim 1 wherein the second holding container comprises an artificial lung circulation loop configured to delivery cell-free media through a lumen side of a hollow fiber artificial lung and wherein the artificial lung circulation loop is configured for delivering oxygenated cell-free media to the second holding container.

7. The system of claim 1 wherein the second holding container comprises one or more sensors for continuous measurement of at least one of pH, oxygen, glucose lactate, ammonia and combinations thereof.

8. The system of claim 1 wherein the first circulation loop comprises one or more probes for measuring pH of media, dissolved oxygen, or a combination thereof of media inside the first holding container and/or wherein the first holding container is configured to continuously monitor a weight of the container which is correlated to a volume of media within the first holding container.

9. A method for high density cell culture support comprising:
concentrating a mass of mammalian cells in a first holding container;
separating the concentrated mass by removing cell-free media from the mass of concentrated mammalian cells in a first circulation loop;
delivering the cell-free media to a second circulation loop;
conditioning the cell-free media in a second holding container in the second circulation loop; removing waste products from the cell free-media in the second circulation loop;
and returning conditioned cell-free media to the first circulation loop.

10. The method of claim 9, wherein conditioning of the cell-free media in the second holding container comprises reoxygenating the cell-free media in a reoxygenating recirculation loop through an artificial lung hollow fiber cartridge device producing conditioned media.

11. The method of claim 10, wherein conditioning further comprises adjusting one or more metabolic parameters comprising pH, glucose, lactate, glutamine, ammonia or combinations thereof

12. The method of claim 9, wherein separating the cells in the first holding container from the media comprises a continuous process and further comprises using centrifugal force in a centrifugation device to remove fractions of cell-free media from the cells in one of a continuous or semi-batch process.

13. The method of claim 10 and returning the conditioned media from the second holding container to the first holding container replacing a volume of waste media being removed from the cells in the centrifugation device.

14. The method of claim 11 and separating the cells in a centrifuge device between the first circulation loop and the second circulation loop by adjusting one or more of retention time of the cells in the centrifuge device, speed of the centrifuge device, rate of a counter elutriating pump, and adjustment of a recirculation rate of the cells to maintain a predetermined level of the media in the first holding container.

15. The method of claim 9 and further comprising:
extracting a sample of the cell-free media from the second holding container;
analyzing one or more of glucose, glutamine, ammonia, lactic acid or osmolarity; and
adjusting one or more of glucose, glutamine, ammonia, lactic acid or osmolarity in response to analysis by adjustment of one or more parameters of a media, glucose or glutamine delivery pumps or a waste removal pump.

16. A combined batch-feed and perfusion culture method comprising:
carrying out a first batch-feed process for seeding cells at a first concentration, allowing the cells to grow to a first volume, and adjusting the cell concentration back to the first concentration by adding conditioned media, and repeating the batch feed process in an integrating vessel until a first selected volume is reached;
engaging a first and second circulation loop to perfuse cells in the integrating vessel and increasing cell density within the first selected volume;
monitoring cell number and cell viability based on samples taken from the first circulation loop through a sampling port in the first integrating vessel;
adding the conditioned media from the second circulation loop for maintaining a constant volume.

17. The method of claim 16 wherein perfusion comprises:
initiating a perfusion cycle including moving cells from the first integrating vessel to a spinning container creating centrifugal force and separating media from the cells to produce cell-free media;
returning cells to the first integrating vessel; and
delivering cell-free media to a second holding container in the second circulation loop for conditioning the cell-free media.

18. The method of claim 16 and further comprising engaging a dialysis loop as a part of the second holding container using a hollow fiber cartridge to remove lactic acid and other metabolic wastes from the cell-free media.

19. The method of claim 16 and continuously monitoring oxygen level of the cell-free media and if the oxygen level falls below a pre-selected set-point, increasing the rate of delivery of the conditioned media concurrently with proportionally increasing a rate of cell removal and waste removal from the first circulation loop.

20. The method of claim 16 and further comprising:
adjusting pH in the second holding container using a proportional-integral-derivative controller control loop for lowering concentration of CO₂ in the artificial lung and replacing the CO₂ with air or N₂ gas; and
when a CO₂ lever reaches zero, adding fresh cell free media or buffer to the second holding container to raise the pH of the cell-free media.
